(19) United States
(12) Patent Application Publication
(10) Pub. No.: US 2004/0087897 A1
(43) Pub. Date: May 6, 2004

(54) Device and method for liquid jet generation

(76) Inventor: Birger Hjertman, Hasselby (SE)

Correspondence Address: DINSMORE & SHOHL, LLP, 1900 CHEMED CENTER, 255 EAST FIFTH STREET, CINCINNATI, OH, 45202, US

(21) Appl. No.: 10/623,252

(22) Filed: Jul. 18, 2003

Related U.S. Application Data
(60) Provisional application No. 60/401,680, filed on Aug. 7, 2002.

(30) Foreign Application Priority Data
Jul. 31, 2002 (SE) 0202350-5

(51) Int. Cl. A61M 5/30

(52) U.S. Cl. 604/70

(57) ABSTRACT

A jet-injector device comprising a) a housing, b) a pressure chamber for a liquid to be ejected attached to or enclosed in the housing, the pressure chamber having at least one opening and at least one movable or collapsible-wall or wall segment and c) a pressurizing mechanism attached to or enclosed in the housing operable to apply, directly or indirectly, force in a force chain between the housing and the wall to pressurize the pressure chamber content for ejection of a liquid jet through said opening, the mechanism comprising at least a force generator and optionally a transmission between the force generator and the wall. The device comprises an in elastic element serially arranged between the force generator and the wall. A method for liquid jet generation comprises the steps of i) applying a primary force, directly or indirectly, on one part of an in-elastic element, ii) applying the pressurizing force by another part of the element, to thereby press the element between the primary force and the pressurizing force and iii) dissipating energy in the element.

### TECHNICAL FIELD

The present invention relates to a jet-injector device comprising a) a housing, b) a pressure chamber for a liquid to be ejected attached to or enclosed in the housing, the pressure chamber having at least one opening and at least one movable or collapsible wall or wall segment and c) a pressurizing-mechanism attached to or enclosed in the housing-operable to apply, directly or indirectly, force in a force chain between the housing and the wall to pressurize the pressure chamber content for ejection of a liquid jet through said opening, the mechanism comprising at least a force generator and optionally a transmission between the force generator and the wall. The invention also relates to a method for generation of a liquid jet.

### BACKGROUND

In jet-injectors for liquid delivery the liquid is given sufficient speed and dimensions to cut through skin or other tissue by the mere momentum and inertia of the jet. Although some jet-injectors are assisted by a short needle or sharp for reduced requirements on the jet penetration power most jet-injectors rely entirely on the jet for penetration and are commonly referred to as needle-free or needle-less injectors. In any case, liquid speeds sufficient for cutting through tissue require that the liquid be pressurized to a high degree in connection with the injection. It is also desirable to have a short rise time for the pressure in order to avoid insufficient jet penetration power, which may result in a “wet-shot” with under-dosing if the liquid is deflected laterally by the skin or in a complete injector failure if the skin displaced away from the injector opening in an uncontrolled manner or if the jet sweeps over the skin. Finally it has long been recognized that it is desirable to have an initial high pressure and speed peak during a short initial penetration phase followed by a lower pressure and speed during the longer injection phase, the latter in order to avoid excessive pain, bruises, over-destruction of tissue and too deep liquid delivery.

There are some inherent problems in the described desired pressure profile for a jet-injector, most of which problems derive from the strong and highly variable pressures involved. Even from a static standpoint the unusually high pressures tend to strain construction materials more than in other injection devices and even small relative variations lead to high nominal differences and force gradients. The difficulties become still more severe when considering dynamic effects. Rapid pressure rise is counteracted by any yield in plastically deformable materials but also builds elastic tension into all hard materials and as the gradients are large and pressure and pressure waves need time to equal out device vibrations and liquid fluctuations will result. Probable overshooting in elastic deformation will remain a cause of variation in later phases of the pressure profile. A second surge of variations is caused by the desired rapid fall off in pressure after the initial penetrating peak and to a lesser degree by variations during the subsequent more or less sustained injection phase. Many parts of the device may contribute and act as source or sink for the variations. In particular the chamber with any supporting structures, pistons gaskets etc. but also the pressurizing mechanism where any solid component may behave similarly but in particular the common force generators, such as mechanical springs or pressurized gas, which are highly elastic and able to support force variations and static and dynamic pressure waves. In fact, the chamber parts and the mechanism parts are designed to exchange forces and the downside of this is that these system parts may resonate and preserve or even amplify undesirable variations. The inventor has been able to measure the anticipated variations both as pressure variations in the pressure chamber as well as force variations at the impact of the jet, and they appear as rapid fluctuations, or “ringing”, superimposed on the slower pressure profile variations. It is believed that this ringing is highly detrimental to the injection process. During the penetration phase the fluctuations reduce the penetrating quality of the jet, perhaps in the same manner as a number of sequential light shots “eating” their way through the target are less effective than a single heavy arrow. During the injection phase liquid stream fluctuations are likely to impose corresponding vibrations in the tissue with increasing problems of delivering the entire dose to the target depth through the cut. Vibrations and fluctuations are also believed to aggravate the sensation of pain as compared to a continuous steady stream. But perhaps the most extreme and devastating consequence of ringing is when the pressure rebound is huge enough to transform an overpressure at the injector orifice into a sub-pressure and hence ejection into aspiration. The inventor has observed, measured and documented such occurrences, at least at the first rebound after the penetrating peak in the pressure profile. If aspiration into the device takes place the sterility cannot any longer be guaranteed and blood borne diseases can be transmitted. Jet-injectors are suitable for mass-injections due to injection speed and the obviation of needle exchange between injections and have since long been used for mass-injection of cattle. Mass-vaccination of humans has been contemplated and tried but abandoned due to occurrences of cross-infection, even when the orifice part is replaced or cleaned. The observed aspiration may give an explanation to the phenomenon and absolutions to the problem may again make jet-injectors a viable option for mass-injection of humans.

Interestingly, it seems that liquid stream fluctuation from jet-injectors is an old observation although the problem seems not to have raised much attention in terms of solutions and improvements. The U.S. Pat. No. 2,762,370 from 1954 notes that liquid is expelled in surges rather than uniformly. However, the inventor attributes the observed problem to natural frequencies in the spring system used and suggests addition of a complementary set of springs having another natural frequency to cancel out the variations. Clearly the proposed remedy is restricted to the spring system and rather introduces more than less elasticity to the system. Otherwise the prior art does not seem to give much guidance to the problem or its solution. Common design principles for reaching the desired overall pressure profile is to enclose the liquid in a strong chamber, or enclosing a soft or fragile chamber in a strong supporting chamber, and pressurizing the chamber by attacking a movable wall of the chamber, e.g. a piston or a membrane, with a mechanism able to provide a strong force and a weaker force suitable for the two phases described. More elaborate devices have separate driving systems for the phases to give a controlled difference in level and duration whereas less complicated devices typically simply allow the mechanism accelerate during a certain dead run distance, creating the peak at impact on the wall followed by a lower sustained equilibrium pressure. Generally such common designs do not at all address the variation or fluctuation problems and neither explicitly or implicitly eliminates any of the problems. This is so because all the necessary prerequisites for the problems to occur are present, namely the presence of system elasticity in combination with the high pressure levels, short rise and fall times etc. in the targeted pressure profile. The WO 01/89614 reference to the present applicant among others describes how circumvention of elasticity in one resilient piston can reduce the total elasticity of a dual-piston cartridge jet-injector system. This is a relevant action for reduction of the device chamber part elasticity to that of a one-piston system but does not eliminate elasticity to the level necessary for avoidance of ringing. Nor does the action influence ringing contributions from the pressurizing mechanism.

The ringing problems described should not be confused with other vibration problems existing for jet-injection devices. For example, various damping means have been suggested for control of recoils effects, caused by relative movements of masses in jet-injectors. The WO 96/28202 reference relates to an injector having an external triggering sleeve movable with respect-to the internal ejection mechanism. A viscous damping medium is located between the external sleeve and the internal mechanism, permitting slow relative movement when pressing the device against the skin but resisting rapid recoil movement when triggered. Clearly such an arrangement is only effective at relative movements between the outer sleeve and the inner mechanism but have no effect on ringing within the mechanism itself. The U.S. Pat. No. 4,722,728 reference describes disc springs arranged between a housing and the main coil spring for the purpose of counteracting bounce or recoil at plunger bottom out impact. Again, such an arrangement is not effective against ringing within the mechanism and is not all in operation during the critical initial and main injection phases. Similarly various damping arrangements have been suggested for control of the movement speed for an injection mechanism, in particular retardation in other than jet-injector devices. The U.S. Pat. No. 6,270,479 reference to the present applicant describes a damper arranged in parallel with an injection mechanism for use in autoinjectors, allowing high injection forces to be applied slowly in non-destructive rates. This is not applicable to jet-injectors where speed is essential for generation of sudden penetrating peaks and high injection rates.

Accordingly there remains a long felt need for control of fluctuations caused by ringing in jet-injectors that has not been met by prior art constructions.

### THE INVENTION GENERALLY

A main object of the present invention is to provide a jet-injector avoiding or ameliorating the above-described problems for existing jet-injectors. A more specific object is to provide a jet-injector with reduced or eliminated ringing, variation or fluctuation problems. Another object is to jet-injectors with reduced or eliminated risks for negative pressures or aspiration. A further object is to offer a jet-injector with reduced or eliminated ringing contributions from both a chamber part and a pressurizing mechanism part. Yet another object is to allow flexible pressure profiles without superimposed ringing over the desired profile pattern. Another object is to offer such jet-injectors still allowing high pressures to be used. Yet another object is to offer jet-injectors permitting fast rise or fall times for the pressure. Still another object is to offer jet-injectors allowing high penetrating pressures followed by lower injection pressures. A further object is offer such jet-injectors utilizing impact for peak generation. Yet other objects are to offer methods for operation of such jet-injectors to obtain the stated advantages.

These objects are reached by the characteristics set forth in the appended patent claims.

Without being bound by theory in this specification of the invention, it is believed that the main factors involved in the cause of ringing phenomenon are system elasticities in combination with high and variable forces. Inelastic materials tend to yield permanently without accumulation of energy or tension that are recoverable, intentionally or unintentionally, later on. In contrast, elastic deformations conserve energy and forces that may be released, intentionally or unintentionally, in later process phases. Certainly some materials may respond in between of fully elastic and fully inelastic behavior but for now it suffices to separate it into ideally inelastic and ideally elastic components. Much inelastic components are not desirable in jet-injectors since they counteract short rise times in pressure. The same goes for elastic materials to the extent they have low coefficients of elasticity and accordingly requires substantial deformation for build-up of counter-force, being the rationale for the earlier described efforts to minimize piston elasticities. However, for the present purposes it is important to observe that the high forces and pressures of jet-injectors makes it necessary to regard as elastic also materials normally regarded as “hard”, such as glass, metal, construction plastics etc. as well as the liquid itself, normally regarded as incompressible. Support for this view is taken from the observed high ringing frequencies and the observed negative pressures, even when the pressure mechanism is not attached to the chamber wall and accordingly being incapable of transmitting aspiration forces. Similar considerations apply for the pressurizing mechanism to the extent it comprises hard materials such as in plungers etc. but the mechanism normally also necessarily comprises components of substantial elasticity in force generating parts, such as in mechanical springs or pressurized gas, which elasticity is intentionally “weak” for the purpose of storing sufficient energy for the entire injection process requirements. In summary, it seems that system elasticity is more or less unavoidable and reduction or elimination of the elasticity is not a general route to ringing suppression. It is further believed that any variations in force or pressure may be the cause of ringing, i.e. not only increasing but also decreasing forces, which in combination with various elasticity tension build-up or fall off, perhaps in combination with movement of masses, may give rise to “overshooting” at compression and decompression. Certainly the main components of the desired pressure profile described may act in this way. But as the forces involved are high and transition times extremely short, momentary equilibrium between applied forces and elasticities cannot be expected but traveling pressure waves, interacting with each other and materials of varying compression properties, will probably also be a main source of ringing initiations. Accordingly neither a full control of pressure equalization can be expected to be a successful general route to ringing suppression.

According to a main aspect of the present invention at least one in-elastic component is inserted somewhere in the force chain between the main force generator and the chamber wall to be affected for the purpose of pressurizing the chamber content. In the normal meaning of an in-elastic element or damper the element shall have parts, the relative displacement of which causes dissipation, consumption or accumulation of at least part of the energy causing the displacement. Use of such an element will enable selective elimination or retardation of detrimental movements while retaining necessary movements. Further, since the element acts in-elastically it will not add to system elasticity but act in quite the opposite manner by eliminating energy without introducing any rebound effects on later system phases, thereby meeting main objectives of not initiating ringing in the system. The element is inserted “serially” in the force chain, not in “parallel”. By in this way not act to retard relative movements between the housing and the mechanism, or between the housing and the chamber wall, the arrangement does not negatively affect the possibilities to impose the desired pressure profile on the system but high flexibility in this respect is retained and the arrangement will be compatible with application of high pressures, rapid pressure rise and fall as well as variations in injection patterns. The serial arrangement means that the element is effective against relative movements between the force generator and the chamber wall, as seen by the element at its point of insertion in the transmission, meaning correction or compensation for any leverage or gear ratio in the transmission. The serial arrangement and the point of insertion means that the element is effective for control of force transfer between the main parts of the jet-injector, namely the pressure chamber part and the pressurizing mechanism part, forming the interface for both the main force exchange and the main differences in elasticity properties. The element may serve to isolate the chamber part and mechanism part from each other, by damping out oscillation movements between these parts, hereby reducing the effective system elasticity available for ringing. The element may act as a filter optimized for preventing exchange of the ringing frequency between the parts but allowing the pressure profile forces, causing less movement, to pass. The element may also serve to reduce existing ringing within each part by damping any variation reaching the interface. More importantly, the element serves to eliminate a major cause of vibrations, namely rebounding or bouncing effects resulting from force variations in the-interface. Any such change may result in bouncing effects if elastic components are involved. The present element eliminates the bouncing by transforming the impulse transfer from elastic to inelastic, of particular importance in connection with the first peak and especially when using a dead-run hit for peak generation. Since the element is effective against relative, rather than absolute, movements it is equally efficient against bouncing or rebound when all the parts in absolute sense continue forward. It may seem counter-intuitive to impose inelastic hits when aiming for high pressures as elastic hits transfer more impulse and dissipate less energy but this is easily compensated for since the difference is insignificant in relation to the total energy turnover in the process. The general influences of the inelastic element are sufficient to significantly reduce or eliminate ringing. Yet elements of this kind are flexible and can be given adapted characteristics for additional advantages, a force profile of its own, one-way properties etc.

Further objects and advantages will be evident form the detailed description of the invention below.

### Definitions

In the absence of explicit statements to the contrary, as used herein expressions like “comprising”, “including”, “having”, “with” and similar terminology shall not be understood to be exclusively restricted to recited element but shall be understood to allow for the presence of further elements as well and shall be understood to cover any element in integral, subdivided or aggregate forms. Similarly, expressions like “connected”, “attached”, “arranged”, “applied”, “between” and similar terminology shall not be understood to cover exclusively direct contact between the recited elements but shall be understood to allow for the presence of one or several intervening elements or structures. The same applies for similar expressions when used for description of forces and actions.

Unless otherwise indicated positional and directional statements given herein, such as “front”, “rear”, “forward”, “rearward”, “axial”, “radial” etc., shall be understood with respect to the force applied to pressurize the liquid, the force being assumed to be applied in the “forward” direction. This direction of the force can be, but must not be, a straight line but the force can be transmitted over changing absolute directions, e.g. in case of various transmission. Neither is the direction necessarily the same as for the ejected jet since the opening for the jet can be oriented freely, even in other directions than the displacement direction for the movable wall.

Elastic device components are roughly referred to as “hard”, “resilient” or “soft” herein. These concepts shall be understood from a functional standpoint in the jet-injector context. The “hard” components shall be understood components or materials, e.g. glass, metal, plastic, liquids etc., that are not designed to yield elastically under the forces involved but may do so to a small degree for reasons explained. The “resilient” components or materials, e.g. rubber, elastomers etc., are designed to yield somewhat, as exemplified by sealing pistons, gaskets etc. The “soft” components, e.g. mechanical or gas springs, are designed to yield, mainly for the purpose of accumulating energy for displacement of the movable wall. Typically the soft components are designed to move, when measured at the movable wall, at least 1, preferably 3 mm and preferably at least 5 mm, whereas the hard and resilient components are designed to move less at the same point of measurement.

Most materials are in between of ideally elastic materials, i.e. with fully reversible behavior, and ideally inelastic materials, i.e. fully irreversible behavior. As a measure of reversible, elastic, behavior for elements of the invention shall be used a “force ratio” Fr/Fa, possibly expressed in percent, where Fa is the force applied to displace the element parts and Fr the force recovered in the opposite direction when the element is released, both preferably measured at forces and over distances similar to or corresponding to the operating conditions in the device when critical, e.g. with the same speed for a speed sensitive element or over the same distance for a non-linear element, and may need to be considered incrementally if not constant. For example, a nearly inelastic element such as a pure viscous damper element will have a force ratio near zero since virtually no return force will be experienced, a memory material will have a finite but low value and a damper device in combination with a spring element can have a lot higher value.

### DETAILED DESCRIPTION

### General

The principles of the present invention can be applied to jet-injectors in broad sense and for varying purposes. As indicated in the introduction jet-injectors are designed to cut through tissue by use of the mere inertia or momentum of the liquid jet. This in contrast to e.g. needle injection where the needle acts to cut through the tissue and any further damage to the tissue is the result of liquid static pressure rather than its inertia. Some jet-injectors a short needle or sharp, e.g. 1-3 mm, assisting in severing the outermost layers, thereby reducing the requirements on the jet although cutting ability is needed also in these cases to reach target depths, e.g. 4-8 mm for subcutaneous and more for intra-muscular injection. The invention can with advantage be used for purely needle-free devices, having the highest requirements for cutting capabilities. Other auxiliary means may be present, e.g. structures for stretching or immobilizing the skin. The injector can be of disposable type intended for a single injection wherein the liquid is pre-filled in the device or charged into the device in connection with the injection moment, e.g. through the opening or possibly form a separate storage chamber being part of the device as exemplified by the WO 01/89614, incorporated by reference herein. The injector may be designed to be re-usable, either for a limited number of injections, e.g. as when charged from a pre-filled permanent storage chamber, or for more or less unlimited number of injections, e.g. when fed from a replaceable storage chamber or supply line, e.g. through the chamber opening, through the collapsible or movable wall or a special conduit into the chamber by assistance of any valve arrangements known as such. For multi-dose injectors it may desirable to make parts of the pressure chamber disposable or the whole pressure chamber as exemplified by WO 01/89613, incorporated by reference herein. Jet-injectors can be designed for a fixed dose or a variable dose, as also exemplified by the latter reference.

The injector described herein may be used for a variety of purposes within and beyond the medical area and for any type of liquid preparations, such as chemicals, compositions or mixtures, delivered for any purpose. For reasons outlined the system have certain special values in connection with medical delivery devices where also the design constraints are more severe than in most other applications. For convenience the invention will be described in terms of this application.

The material to be delivered is a liquid, including materials behaving as liquids such as emulsions or suspensions. These observations relate to the final preparation whereas other components, notably solids, may be present before final preparation. The nature of chamber content shall also be understood to include, medical in broad terms and to embrace for example natural components and body fluids pre-filled or drawn into the chamber although most commonly the medical is factory prepared.

The invention will be described mainly with reference to the components of the device as initially stated.

### The Housing

The housing shall be understood in broad sense and basically as a point of reference for positional and directional statements for other parts and components. It is preferred, however, that the housing also actually enclose at least the mechanisms of the device and leave exposed mainly the parts that should be accessible to the user, such as arming, triggering and cocking controls as well as means for convenient use such as grip, handling or support structures. The chamber can be integral with the housing or can be attached to the housing in such a manner that it is exposed or so that the housing also confines the chamber, the latter especially if the housing supports the chamber in respect of pressure resistance. As indicated the chamber may be permanent or replaceable. Replacement of chambers may be facilitated by any known separation or openable arrangement or any connection or attachment, e.g. threads, bayonet couplings, ball locks, etc. In case the pressure chamber is charged form a separate storage container or supply line the housing may enclose or incorporate attachments for such features.

### The Pressure Chamber

The pressure chamber shall be able to sustain the ejection pressure, either by itself or when assisted by support structures and shall be designed to allow pressurization of its liquid content. Any contemplated or known chamber type meeting these demands can be used. It may be a monolithic, integral or composite structure. The movable wall may need adaptation to each type of pressure chamber. For example, the chamber may be an entirely flexible sack, which is externally supported. This is exemplified by U.S. Pat. No. 2,642,062 wherein is described a flexible sack collapsible from its unsupported rear end, which acts as movable wall by being exposed to a hydraulic medium pressurized with a spring and valve arrangement. The U.S. Pat. No. 3,308,818 describes a supporting enclosure confining a flexible sack and a pyrotechnical propellant wherein the propellant directly affects the sack for compression. The U.S. Pat. No. 5,026,343 reference describes a more rigid ampoule with a collapsible rear wall, which is collapsed by a spring driven plunger arrangement. The chamber can also be a generally rigid structure with a separate part acting as a movable wall, e.g. as in the common cylinder/piston kind of arrangement, or generally vessel/wall wherein vessel shape and movable wall have to be mutually adapted. The vessel may be designed most freely when the wall is a flexible or oversized membrane or diaphragm able to adapt by movement or reshaping to vessel internal surfaces. Preferably, however, the vessel has a substantially constant internal cross-section, with a similarly constant vessel axis, between front and rear parts giving a generally tube-shaped vessel, and most preferably the cross-section is of the common circular type giving a substantially cylindrical vessel. The movable wall is then preferably a substantially shape-permanent, although possibly elastic, body sealingly adapted to the internal vessel surface and preferably of the plunger type having sufficient length to self-stabilize against tumbling during-travel along the vessel. Rigid chambers are typically manufactured from metal, glass or preferably a rigid plastic like polycarbonate. As indicated, the pressure chamber may have any additional feature for secondary purposes, e.g. inlets channels, inlet and/or outlet valves etc. for single or repeated filling or arrangements for attachment or replacement. The chamber may also be designed as a dual or multi compartment chamber, to be described below. In many instances it is preferred, however, that such preparatory steps are made before charging a preparation into the pressure chamber and in such instances the multi compartment chamber can be separate from the pressure chamber.

Dual or multi compartment chamber types for injection devices are known e.g. for preparations demanding a mixing of two or more components or precursors before administration. The components are kept separated by one or more intermediate walls of different known designs, which walls divide the chamber into several compartments, for cylinder type chambers sometimes placed parallel along cylinder axis but most commonly in stacked relationship along the cylinder axis. Unification of the components may take place by breaking, penetrating or opening a valve construction in the intermediate walls. In another known design the intermediate wall or walls are of the piston type and flow communication between the compartments is accomplished by moving the piston to a by-pass section where the interior wall has a piston deforming section or one or several enlarged sections or repeated circumferential grooves and lands in a manner allowing by-flow of rear compartment content into front compartment at displacement of the rear movable wall. The chambers may contain gas, liquid or solids. Generally at least one liquid is present. Most commonly in pharmaceutical applications only two compartments are present and typically contains one liquid and one solid, the latter being dissolved and reconstituted during the mixing operation.

The pressure chamber has at least one opening, also referred to as an orifice, through which the preparation pass during the main jet delivery operation of the device. It is also known to use the opening for flow to the chamber, e.g. at preparation steps such as filling, mixing or dissolution in the container, during which operations the opening need to be present. It is possible and even in many situations preferred that certain device operations, such as initiation, takes place before communication has been established and the opening requirement shall then be considered satisfied by the preparation arrangements for creating the communication such as the presence of a removable closure or a pierceable or rupturable part. The opening may also be equipped with a valve arrangement, preferably biased to a closed position and opened in connection with the injection, either manually or by the applied pressure. Size openings shall be sufficiently large to give reliable penetration but not so large as to cause too much tissue damage. Typical opening sizes are more than 0.01 mm, preferably more than 0.03 mm and most preferably more than 0.06 mm and less than 1 mm, preferably less than 0.6 mm and most preferably less than 0.3 mm. Preferably only one jet is formed but it is also possible to provide several openings for generation of several jets. Commonly the opening axis is parallel or even coaxial with the main direction for the wall movement during chamber pressurization but it is also possible with an offset or angled arrangement, e.g. for better access in treatment in body cavities like in dental applications.

Typical maximum pressures in the pressure chamber are in general above 25 atm (2,5 MPa), often above 50 atm (5 MPa) or above 100 atm (10 MPa). Normally the pressures are below 1000 atm (100 MPa), often below 800 atm (80 MPa) or below 500 atm (50 MPa).

### The Pressurizing Mechanism

The pressurizing mechanism shall be arranged to apply force on the pressure chamber movable or collapsible wall, being the interface between the liquid and the force chain. Accordingly the mechanism shall comprise a “force generator”. Since the force shall be applied over a certain distance the force generator can also be regarded as an “energy source” and these concepts will be used interchangeably herein. Also the concepts “force” and “pressure” will be used interchangeably herein dependent on context, e.g. the mechanism might better be understood in terms of forces whereas the force applied to the chamber might better be understood in terms of pressure in the normal meaning of force per area.

The force generator may utilize manual force for movement of the wall but normally stored energy in the meaning of other than manual energy is used as energy source. This shall not exclude that the force generator is cocked, or the energy source charged, by use of manual force or energy respectively, which arrangement is quite common e.g. when using mechanical springs and sometimes when using gas pressure. Otherwise any kind of stored energy can be used in the force generator, e.g. compressed mechanical springs, compressed gas or propellants, pyrotechnically, chemically or electrochemically released gas, electro-mechanical energy, as exemplified by U.S. Pat. No. 5,116,313, etc. The force generator can be separate form the mere injection device, e.g. placed remote and transmitted via a transmission link, as illustrated by the last-mentioned reference, but is preferably included in the housing for truly portable and hand held devices Cocking may take place with the same or different kinds of energy and forces. Similarly cocking arrangements may be included in the housing of a portable device but can also be separate from the device, as illustrated by U.S. Pat. No. 5,704,911 and U.S. Pat. No. 3,815,594. More than one force generator may be present, either acting in concert like a plurality of springs acting as a unit as in the mentioned U.S. Pat. No. 2762370 or designed to act during different phases as in the mentioned U.S. Pat. No. 5,116,313.

The force generator may act directly on the wall, e.g. a spring or a gas as exemplified, but optionally the device may include a transmission between at least a part of the force generator and at least a part of the wall, being the part in contact with the liquid. The transmission can be a simple mechanical connection, e.g. a plunger rod affecting a wall in the form of a membrane or a piston. The transmission may act to redirect the force via for example a mechanical link arrangement or a hydraulic channel, e.g. to affect the general layout of the device for example as exemplified in references mentioned. The transmission may serve to transform the force, e.g. change force profile over distance, gear down or, more commonly in jet-injectors, gear up the force, e.g. by a mechanical gear, link or lever arrangement or in gas and hydraulic transmission by a change in surface areas of plungers and pistons in known manners. When more than one force generator is present each may have transmission components of its own, like the plungers of different diameter in the mentioned U.S. Pat. No. 2,762,370 and U.S. Pat. No. 5,116,313, and/or common transmission components, like the hydraulic link in the latter reference.

### Control System

The injector may also include a control system for sequencing its various operation phases, in particular the penetrating phase and the injection phase when present. As indicated the penetrating peak can be generated simply by impact of an accelerated mass, e.g. contained in the force generator, a transmission or the element of the present invention. In this case the control system need only secure that start of the force generator movement takes place from a position providing a gap somewhere in the force chain between force generator and the wall where sufficient mass exists behind the gap and preferably as far forwards, with respect to wall movement, as possible and most preferably directly between the wall and the force generator or the transmission as the case may be. This gap may be adjustable, e.g. to correct for different penetration requirements or preparation viscosities, and the start point may be adjustable, e.g. to allow for different start positions for the movable wall for example in case of accommodation of different chamber sizes or filling degrees. Alternatively the penetrating pressure peak can be generated without need for a gap, even by use of one and the same force generator or force generator assembly. This can be done for example by applying a more or less similar force first on a smaller area of the wall and then on a larger surface of the wall, as exemplified, and the control system may then be arranged to engage the larger surface after a given operation distance for the smaller surface. Still with use of one force generator a force transformation can occur, e.g. by any of the means discussed above in connection with transmissions, by increasing the force for the penetration phase or lowering it for the injection phase, and the control system may then be designed enable or disable the transformation between the phases, e.g. by mechanical locks or valves for fluids. Still another alternative is to use separate force generator systems for the two phases, which force generator systems can be of similar or different nature, and the control system may then be designed to start or enable the injection force generator system, and preferably also stop or disable the penetration force generator system, at the end of the penetration phase, which may require similar mechanical locks or valves for fluids. Other means can be used by the control system such as processor-controlled operation. The control points for the actions described can vary and for example be after a predetermined run for the force generator or the movable wall or at a predetermined point with respect to the housing. The described alternatives can also be used in various combinations.

The control system may also include other features. Certainly the control system may include a manual control forming the interface between user and actual mechanism. In case of stored energy the control may take the form of a trigger, releasing e.g. a valve or a mechanical lock, enabling the force generator for action. Another common trigger arrangement for jet injectors is a trigger that acts when a certain pressure is exerted against the target, as has been exemplified. The manual control may include common safety details such as an arming lock or command requirements making the device child proof. An additional control may be a dose setting mechanism. Doses of different volumes can be drawn or charged into the pressure chamber under simultaneous rearward movement of the movable wall. Alternatively the pressure chamber may be charged with varying volumes with fixed movable wall followed by de-aeration of the pressure chamber by forward movement of the wall. For these purposes the control system may comprise features for the charging and for adjustment of the force generating mechanism or transmission to corresponding variable start positions. The mentioned WO 01/89613 reference describes various constructions for such alternatives. It is also possible to control dose volume by only partial emptying the pressure chamber and the control system may then need an adjustable stop arrangement for the mechanism. Also known as such in the art are controls for adjusting the force from the force generator and arrangement for facilitating cocking of the force generator.

### The Element

According to the present invention at least one in-elastic element shall be utilized for the purposes outlined, e.g. to avoid ringing generation or to damp out generated ringing. The “in-elastic” property means that the element shall be able to remove work, i.e. force times way, energy forms applied to at least two parts of the element displaceable, externally or internally, with respect to each other. The removal shall be at least partially irreversible, not to act elastically, and any principle for irreversible removal can be used, e.g. one-way consumption or accumulation of the energy, although in most instances the common irreversible principle of dissipation of the energy as heat is sufficient and preferred. For the latter principle any “friction” mechanism in broad sense can be used for dissipation of energy. The friction mechanism can be based on electric resistance losses, e.g. by induction of stray currents or in a resistive conduit for best adjustment and control. The friction can be generated in common viscous dampers, in the meaning of having a fluid, gas or preferably a liquid, arranged to pass a flow constriction or between shear surfaces during displacement of its parts. Viscous dampers are flexible and may act immediately upon displacement of its parts without delay or dead run. Viscous dampers, or dash pots, are well known components as such and may take a variety of forms, e.g. axial, as exemplified by piston/cylinder types in which the fluid passes constrictions in or around the piston or in controlled shunts, or rotational, as exemplified by impellers rotating in a fluid under generation of shear forces. Friction can be mechanically generated e.g. between compressed friction surfaces as in any principle known from brake systems, e.g. allowing unlimited control of friction force and movement distance. A special kind of mechanical friction can also be provided by structures being deformable in an in-elastic manner at part displacement, often providing the property of automatically giving a response force growing with deformation and hence also with distance. The deformation can be permanent as in collapsible cavity grid structures, e.g. honeycomb structures, which may be useful in disposable devices, or reversible when such structures utilize memory or foam materials. Reusable, albeit not reversible, such deformable materials are for example particle filled containers wherein work is believed to be dissipated as heat by random collisions and friction between the particles, such arrangements additionally able to provide mass and often able to be deformed in more than on direction. The container can be soft or collapsible for immediate action upon impact, e.g. as in known sand bags, but can preferably be shape permanent cavities, or external borders, to allow for partial filling, believed to give more reproducible results and possibly a slightly delayed action consistent with precompression of elastic components of the system. These elements can with preference be used in connection with impact effects such as a gap. From a design standpoint such elements are shape permanent and dislocation of its mass center acts as an internal displacement of its parts. Such elements are known as such from so-called “dead-blow” hammers and have with success been used for the purposes of the present invention. The cavity can be equipped with at least one movable mass in which case it is preferred that the weight is guided or connected to the cavity with friction, e.g. on friction rails, guiding pins or immersed in liquid. However, a preferred type is filled in gas with numerous metal particles, e.g. shots, to a high but partial filling degree, meaning that the particles shall be able to move, e.g. to more than 50, preferably more than 65 and most preferably more than 80 percent bulk volume but to less than 100, preferably less than 99 and most preferably less than 95 percent bulk volume. Other in-elastic elements than those exemplified above can be used when selected or configured according to the considerations given herein.

The elements described, or in-elastic elements generally, can have different characteristics with respect to counterforce as function of a number of variables. With respect to speed of relative movement of the element parts, the element may respond with constant force, i.e. also constant energy work consumption over unit distance, e.g. typical for mechanical surface friction elements, for example useful for uniform transfer of impulse, i.e. force times time, to provoke minimum system elastic build-up. The force may be directly proportional to speed, e.g. for a viscous damper operating with laminar flow, or proportional to speed squared, or higher increase rates, for viscous dampers operating with turbulent flow, for example for efficient damping out of existing ringing or for increased stiffness. These general relations or functions will also apply for energy work consumption per distance unit. The general relationships can certainly also-be-modified and altered by active control or by design. For example the force can be made more or less dependent on velocity by arrangement or control of valves for fluid flow in viscous dampers or can be made dependent over distance traveled by variations of contact pressure in friction elements, e.g. as in wedge arrangements, or variations in slit sizes over distance in viscous dampers. Such modifications can also make the element response asymmetrical with respect to movement direction, as known as such in the art, e.g. giving the response mentioned only in one direction but giving no response, different response or stiff response in the opposite direction, e.g. by use of one-way valves in viscous dampers, ratchet arrangements etc., for example to prevent aspiration, rebound etc. or to assist in resetting the device mechanism, possibly with releasable arrangements. Various such damper designs and modifications are known as such in the art, albeit not for the present purposes. Certainly the characteristics discussed can be permanently set or can be adjustable.

As indicated some of the elements, e.g. inelastically deformable materials, are able to absorb energy in more than one direction, it is generally sufficient for the present purposes that the damper can absorb energy in one direction, including when desired both forward and rearward motion. Still a transmission may be needed, e.g. to transform a linear movement in the device into a circular movement in rotational viscous dampers, to permit space-conserving repositioning of the damper or to allow for a force modifying lever arrangement.

As will be further discussed below the element is inserted serially into the force chain from the force generator to the movable wall of the pressure chamber, meaning that the static and dynamic forces transmitted in the force chain affect the element, potentially consuming part of the movement length for the force generator mechanism, representing a “lost distance” that may require an added movement distance at mechanism design. For the present purposes such a movement length is typically at least 1 mm, preferably at least 2 mm and most preferably at least 3 mm. These values can also be said to represent a minimum stroke length for the element, i.e. the minimum displacement of its parts. The maximum stroke length is less critical since often it need not to be utilized in full. The stroke length loss may need to be controlled. Since the jet-injection process is short it is possible to use even elements of unlimited stroke length, e.g. brake type elements, or elements of limited stroke length that shall-not bottom out, e.g. cylinder/piston type elements, as the process may terminate before too long or full stroke length has been consumed. It is often preferred, however, to provide a stroke length limiter. The limiter may be a simple stop surface, added or occurring naturally as in cylinder/piston type elements, but it is in general preferred to use limiters that provides an over length increasing counter-force, e.g. to limit stroke length loss without imposing impact shocks to the system or to allow the element to come into equilibrium with the force applied in the force chain. As indicated, some of the exemplified elements, e.g. inelastically deformable materials, inherently provide this property. It may also be preferred to use a limiter that provides such an increasing counter-force in an repeatable fashion, e.g. an elastic limiter connected in parallel with the in-elastic element. Such an elastic limiter can be for example any of the elastic means suggested for the force generator, e.g. mechanical or gas springs, and preferably such a limiter being “soft” in the sense of acting over a significant distance. This combination arrangement, with an in-elastic element in parallel with an elastic limiter, may serve to allow the combination to come into equilibrium with different and varying forces in the force chain. The combination may also serve as a “filter” against certain, e.g. ringing, frequencies by allowing the transmission to oscillate around an equilibrium force under simultaneous damping of the movement in the element. The combination may then be optimized for filtering out the desired oscillation characteristics. The limiter may also act to reset the element to an initial uncompressed state, e.g. to facilitate repeated use. When a limiter is used a typical maximum stroke length can be less than 30 mm, preferably less than 25 mm and most preferably less than 20 mm. The mechanism movement and element stroke lengths described refer to distances at the movable wall whereas the distances may be different at other parts of the force chain, e.g. due to transmission components, and also different internally in the element.

The element may have an internal gear or leverage ratio between the oscillation amplitudes applied to the element and the actual stroke length in the element. This can be used to reduce the internal stroke lengths but are preferably used to amplify the internal stroke lengths, which serves to make damping more reproducible and efficient and to facilitate design of an elastic counterforce component, since the distance amplitudes may be small in the force chain in spite of high forces. The ratio can be realized by any of the means mentioned in connection with the transmission, e.g. lever, gear or hydraulic surface ratios. Preferably the stroke length is amplified to more than 2, more preferably to more than 5 and most preferably to more than 10 times the applied lengths.

It is clear that useful element may be substantially entirely inelastic, e.g. the collapsible elements, or incorporate an elastic component as well, e.g. the combination elements for equilibrium purposes, making it difficult to give general directions for the elasticity property of the element in whole. However, as guidelines the total element shall act as an inelastic component and accordingly have a force ratio, as defined, of clearly less than 100%, such as below 90%, below 75%, below 50% and even belw 25%. Elements without intentional elastic components can with preference have force ratio values even lower than 25%, such as below 10%, below 5% and most preferably below 1%. These lower values may also apply to the inelastic component in a combination element, whereas the elastic component should have a higher force ratio values than the inelastic component, such as above 25%, above 50%, above 75% and even above 90%. The component properties cannot always be separated, e.g. as for a foam material with intermediate behavior, and the above values for the total element should then be relied on. Wihen determining the force ratio it is often sufficient to consider it over a part of the element stroke length provided this part is relevnt for device in operation.

### Element Use

According to the present invention at least one element shall be included in the force chain from the force generator to the movable wall. Certainly a front part of an element can act as the movable wall although it is generally preferred to make the wall separate from and preferably not attached to the element. The element shall be arranged in “series” in the force chain, meaning that its movable parts are affected by the force transmitted in the force chain at the point of element insertion, e.g. squeezed together by the force driving the wall forwards or generally by the overall pressure profile applied plus the superimposed ringing at this point. The serial insertion does not negatively affect the movement speed for the force generator or the forces transmitted, which is essential for necessary quick operations in jet-injectors. All this in contrast to a “parallel” arrangement wherein the element is affected by the force applied between a support, notably the housing, and the point of insertion in the force chain, which arrangement will retard the speed of the force generator reactions. Certain spring types, e.g. packages of leaf springs or braided coil springs, have inherent friction, acting at spring movement, but such friction is arranged in parallel with the elastic spring component in the present sense by acting against the spring movement as such. The same applies for constrictions in hydraulic or pneumatic transmission components in a force chain, especially if the constriction is fixed with respect to the housing.

For reasons discussed the-serial arrangement of the element does not exclude that other functional units are connected in parallel with the element, e.g. the elastic limiter discussed above since also such a unit or limiter will not retard, but move with, the movements in the force chain. It is also possible to include an in elastic element in parallel as an auxiliary functional unit, preferably then for secondary purposes like retardation under an initial initiation phase, e.g. preparation mixing, prevention or retardation of rearward motion by a oneway element, e.g. to avoid aspiration, or speed control of a cocking movement.

One or more elements can be inserted at one or more positions in the force chain to meet various objectives to be discussed in general terms below.

An element may be arranged serially in the rearmost part of the force chain between the housing and the force generator. Such an element will be affected by the force applied by the force generator to the force chain and the element is preferably a complementary arrangement as described being able of sustaining and coming into equilibrium with this force. Such an element may be active e.g. for damping out ringing oscillations reaching and potentially being reflected at this end of the force chain. The element may also act against ringing vibrations otherwise resulting from the sudden release of the force generator mechanism and corresponding accelerations and displacement of masses, even if the element may not be active against the recoil as such.

A similar element may also be arranged within the force generator “soft” parts, e.g. between parts of similar or different serially arranged spring elements, e.g. for damping out ringing or resonance proceeding within the spring system. One or several similar or different elements may also be positioned in one, part of or all of several spring elements arranged in parallel in the force generator, e.g. for the purpose of additionally providing variations in the oscillation characteristics for the different spring parts to counteract or defeat resonance oscillations.

However, instead of positioning the element or elements within the force generator soft parts according to the preceding paragraph and for similar purposes, it is preferred to position the element or elements in front of at least the soft, e.g. spring, parts of the force generator. This often facilitates the design, is applicable to most force generator types, including e.g. gas springs, and avoids adding mass to the soft parts of the force generator. A positioning in the interface between hard and soft force chain parts, having different elasticity properties as described in the introduction, also utilizes the element to a maximum for both sides of the interface.

As indicated a transmission may be present between the force generator and the movable wall. An element can be positioned within the transmission, e.g. to damp out any oscillations at the source of origin for example at play and tolerances. In case the transmission comprises soft components, e.g. counter-springs, an element can be positioned in the interface in front of the soft component. Generally it is preferred to keep all soft elasticity components on one side of an element, especially if only one element is present in the force chain. Incorporating some hard elasticity component on the same side is less critical and accordingly the element can be positioned in front of the transmission if desired, e.g. with regard to design considerations.

A preferred position for an element is in the front-most part of the force chain, at or close to the pressure chamber movable wall, or a piston rod for the wall if necessary for access. Such an arrangement minimizes system elasticity components in front of the mechanism to those more or less unavoidable for the pressure chamber and its auxiliary parts and an element here can be used for example for reaching the fastest possible reaction against unavoidable elastic responses. At this point it may also be difficult to avoid at least small gaps, e.g. between the mechanism and a replaceable pressure chamber, or compressible parts, e.g. piston type movable wall, potentially giving impact and rebound effects to be further discussed below. If desired an element at this point can be complementary element more to the rear for purposes discussed earlier.

In the element uses above reference have been made mainly to the damping of ringing or similar oscillations in the system. For such purposes it is possible to damp out only one, or a limited number of oscillations, e.g. only the first down-surge in pressure after the penetrating peak of the pressure profile. A “damper element” for the purpose of damping out existing ringing it is possible to utilize also elements single use elements, for example the deformable elements discussed. One-way elements, as discussed, can be used if it is desirable to damp out changes in only one direction, e.g. pressure decreases generally. Normally, however, it is desirable to damp out ringing type oscillations in general for which purpose it is preferred to utilize elements allowing repeated relative movements between the element parts to follow the oscillations and most preferably the combination arrangements discussed, allowing such movements superimposed on an equilibrium position corresponding to the main force component of the pressure profile. It is clear that the element should be optimized for removing the ringing but allowing-the slower pressure variations to pass.

Another object of the present invention is to avoid generation of, rather than defeating existing, ringing and other oscillations. As indicated in the introduction it is believed that any change in force or pressure may be the cause of unwanted oscillations. As also indicated some causes may be design related, such as play and tolerances potentially giving impact effects. Others are unavoidable as being part of a desired pressure profile, notably the rapid initial pressure rise for penetration purposes as well as a following rapid pressure drop to non-destructive injection pressures. The cause of ringing due to force changes can be thought of as reflections of rebound effects. Even if all parts in the force chain are in physical contact, and accordingly no impact effects are involved, rebound takes place by build-up of positive or negative compression in system elastic components, which compressions become active when the forces in the force chain changes. This may be a significant factor for example in connection with such injectors types, as exemplified, where the penetration and injection pressures are created by force application on a smaller and a larger surface respectively or by sequential activation of different or additional spring systems. Certainly gaps in the force chain will be the source of additional rebound due to impact effects. This may be a significant factor for example in connection with the common injector types utilizing a mechanism dead run and impact for generation of the penetrating pressure peak. In all of these situations an inelastic element may serve to reduce or eliminate the rebound effect. The effect obtained can be thought of as an equivalent to a collision between in elastic bodies where the bodies tend to stop or continue their movement together, independent of their initial mass and velocity.

An “impact element” for this purpose of avoiding rebound effects the same type of elements can be used as mentioned in connection with damping out existing ringing, if adapted to the target forces involved. For example, the combination elements can be used that are able to come into equilibrium with and oscillate around the force in the force chain, especially when aiming for action against repeated force changes, e.g. in more complicated pressure profiles. However, other element types can also be used, such as the other mentioned or in particular elements of deformable materials. When preventing ringing generation it becomes possible to act against a specific cause, e.g. an impact gap present in the force chain. In many jet-injectors the main source of rebound and ringing is the generation of the initial penetration peak and it may be sufficient to design for this peak only. Both the first up-surge and the following down-surge down to a lower injection pressure are desirable pressure profile characteristics that should not be suppressed. Accordingly it might be sufficient to suppress overshooting in the down-surge. For purposes like this it is possible to utilize also elements that act in one direction or act only once, such as the deformable or collapsible element types mentioned such as the fixed cavity types. It is believed that the element at least in part acts to stabilize the nominal value of the force generator by extending the time for impulse transfer to more than the duration of the down-surge. At least one element should be present in the force chain for such purposes and preferably be located in front of the soft part of the force generator, preferably also in or in front of any transmission and most preferably close to the front of the mechanism, i.e. near the wall or its optional piston rod, to minimize system elasticity participating in the rebound.

### Design Considerations

It is preferred that the elements provide an initial counter-force, e.g. not to counteract a rapid build-up of the initial peak or not unnecessarily extend the lost distance. It is preferred that the element does not yield substantially below forces corresponding to at least 10% of the maximum force value in the initial peak, preferably not below 20% and most preferably not below 30% of this force. The element should yield below the maximum peak value, preferably below 90% and most preferably below 80% of this value. A preferred rough target is the force where the force line for the main spring intersects with the initial peak. These characteristics can be provided by any of the means discussed. For example a collapsible element can be given a corresponding design resistance against initial deformation, a particle filled shape permanent cavity element inherently has these properties and a combination element can be submitted to an initial corresponding equilibrium force, e.g. by before triggering being compressed by the force generator for example by locking the force chain in front of the combination element.

It is further preferred that the elements are designed to be able to increase their counterforce, e.g. to allow the highest values and speeds in the peak and to be active against oscillations. It is preferred that the element can stiffen at least to forces corresponding to the maximum value in the peak although it is also possible to control that peak value by designing the elements to yield substantially at a desired design value so as to extend a spring force generator to lower forces. As discussed many collapsible elements are naturally stiffening or can be made so and combination element can be given a sufficiently strong elastic component to enable equilibriums also at the desired increased counterforce.

It is further preferred that the counterforce of the inelastic element component, disregarding any elastic element component, of the elements lies in between “weak” and “strong”. If the inelastic, or friction resistance, force is too weak the dissipated energy will be too small due to a small “force factor” in the force times way energy form and if it is too strong the dissipated energy again will also be small due to a small “way factor” in the force times way energy form. As an indication, a suitable balance between weak and strong inelastic resistance may refer to the maximum pressure profile force during operation, e.g. the said maximum peak value. The resistance force can be above 10%, preferably above 20% and most preferably above 30% of the maximum force and can be below 90%, preferably below 80% and most preferably below 60% of the maximum force. Still better is if these limits apply relative the momentary force transmitted, rather than the peak value, i.e. that the inelastic counterforce component adapts to variations in the force transmitted, e.g. by use of the inherently stiffening elements or force regulating components in viscous dampers, which will optimize damping. It should be noted that the element total counterforce could be higher than mentioned here due to contributions from elastic components present.

### SUMMARY OF THE DRAWINGS

FIG. 1 illustrates schematically a hypothetical jet-injector according to the invention and its various components.

FIG. 2 illustrates schematically a typical pressure profile curve for an injector operating with an impact gap.

FIG. 3 illustrates schematically a typical pressure profile curve for an injector operating without an impact gap.

FIGS. 4 to 7 illustrate schematically various collapsible in-elastic elements.

FIGS. 8 to 10 illustrate schematically the principles of a combination element, wherein FIG. 10 illustrates schematically a combination element with arrangements for stroke length amplification with respect to lost distance.

FIGS. 11A and 11B show two pressure profile samples of a prior art jet-injector.

FIGS. 12A and 12B show two pressure profile samples of the jet-injector of the same jet-injector as used in FIG. 11 modified with a collapsible element.

### DESCRIPTION OF DRAWINGS

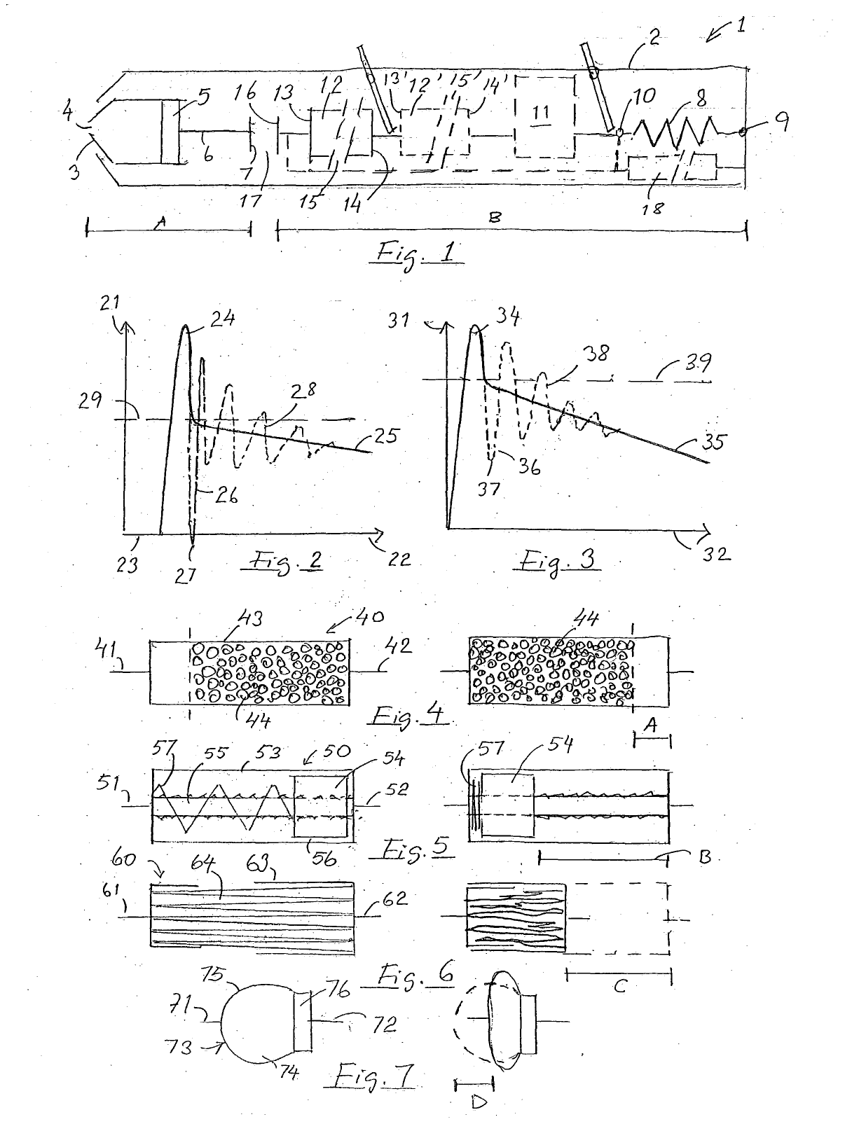

FIG. 1 illustrates schematically a hypothetical jet-injector according to the invention and its various components. The injector 1 comprises a housing 2 to which is attached a pressure chamber 3 having an opening 4 for ejection of the liquid, a movable wall 5 in the form of a piston and a piston rod 6 for displacement of the wall to be affected at its rod end 7. These parts can be regarded as the pressure chamber part of the device and are illustrated as section A of the device. Section B of the device can be said to represent the pressurizing mechanism or force chain of the device. This part comprises a pressurizing spring 8, e.g. of any type mentioned, with its rear end 9 attached to the housing and its front end 10 attached to the further force chain, an optional transmission 11 component, e.g. a gear up or gear down mechanism, an in-elastic element 12, having a front part 13 and a rear part 14, which are axially movable with respect to one another as illustrated at 15, and optionally a second element 12′, similarly having a front part 13′ and a rear part 14′ movable as illustrated at 15′. The force chain ends at a pressure surface 16 arranged to apply force on piston rod end 7. Between the pressure surface 16 and the piston rod end 7 there is an optional gap 17 useful if an impact is desired for generation of a first penetrating peak pressure. Alternatively there is no gap by the spring is dimensioned to give the necessary pressure without impact. Element 12, and optional element 12′, are serially arranged in the force chain, meaning that the front part, 13 and 13′, and their rear part, 14 and 14′, are connected directly to the neighboring parts in the force chain, meaning that they are part of the force transmission force chain, e.g. compressed by compressive forces in the force chain an vice versa, and are submitted to the same forces as applied to the pressure chamber. For illustration purposes an alternative parallel arrangement of an element 18 is shown, the ends of which element is connected over the spring 8 by being attached to the housing 2 and the front end 10 of the spring, or alternatively over the entire mechanism by being attached to the end 16 of the force chain. Such an element will retard the movement of the spring or mechanism and the element will be submitted to the force of the spring, i.e. not the same as the force applied to the movable wall, which is the spring force less the friction force of the element. Further, unless inverted, the element will be subjected to stretching forces at compressive transmitted forces and vice versa. Finally a trigger 19 is shown at the front end 10 of the spring. This position is suitable for example if it is not desirable to subject the elements 12 and 12′ to the static spring forces before triggering, e.g. if any of the elements is collapsible under these forces. However, the opposite might be desirable for example to let a combination element, as described, come into equilibrium with the force before triggering. Trigger position 19′ is between elements 12 and 12′ and can be used for example if element 12 is collapsible and element 12′ is an equilibrium element.

FIG. 2 illustrates schematically a typical pressure profile curve for an injector operating with an impact gap. On the vertical axis 21 is shown the pressure in the pressure chamber or the force applied to the movable wall whereas on the horizontal axis 22 is shown the extension of the mechanism at the wall although the curves will be much the same if time is used instead. It should be noted that in case an element is present in the force chain the mechanism extension to the rear of the element might be longer, up to the element stroke length, than the extension in front of the element. The solid curve illustrates a target profile and the dotted curve illustrates a superimposed ringing pattern. Beginning with the solid curve, after an initial delay 23 due to passage of the gap, a rapid pressure rise to a peak 24 is obtained at mechanism impact on the movable wall or its piston rod. When the impact effect is over the pressure returns to a value curve 25 represented by the spring characteristics and falling off with further spring extension., e.g. more or less to a linear function for a mechanical spring or an inverse function for a pressurized gas. However, the dotted line illustrates a frequently encountered deviation from the ideal pattern. When the pressure falls off from the peak value it overshoots the spring curve 25 into a strong down-surge 26, which may even reach negative pressure values as illustrated at 27. The pressure variations continue to over-shoot the spring curve 25 in a diminishing ringing pattern 28. Line 29, roughly at the intersection between the initial pressure rise and the spring curve 25, illustrates a suitable pressure level where an element inserted for avoiding ringing generation can begin to yield and preferably continue to yield under at least a stroke length or duration sufficient for completion of the peak 24 and return to the spring line 25. For reasons outlined such an element shall preferably be stiff or stiffen during yield in order to be able to transmit the necessary forces in the peak. An element inserted to defeat existing ringing can be an element able to come into equilibrium at forces represented by line 25 and able to oscillate under damping around these force values with force amplitudes being at least part of the non-damped curve 28 and preferably at least part of the extreme values at 26 and 27. Although the element with preference can be able to perform these extreme amplitudes this is not entirely necessary as the ringing amplitudes will be much less when damped.

FIG. 3 illustrates schematically a typical pressure profile curve for an injector operating without-an-impact gap. The axes 31 and 32 and the solid and dotted curves have the same general meaning as in FIG. 2. When the pressurizing mechanism operates without a gap and in initial contact with the movable wall the pressure rise will start almost immediately at 33 upon triggering. A peak 34 that overshoots the spring curve 35 will be obtained also in this case, due to the dynamic effects discussed, although the peak will be less high over the spring curve 35 in the absence of an impact after dead run. In order to have the same peak value as when using a gap it might be necessary to use a stronger spring giving higher maximum forces as illustrated when comparing spring curves 35 and 25. Further, in order to end up with comparable non-destructive final injection pressures the spring curve 35 can with preference fall off in force more steeply than in spring curve 25, which can be obtained for example by a stronger but shorter mechanical spring, a gas spring of higher pressure but less volume or greater driving surface etc. With regard to the ringing curve this generally less pronounced than when using an impact, here illustrated will a smaller sown-surge 36, a less minimum value 37 and smaller oscillations 38. As in FIG. 2, line 39 represents a suitable yield force for an element inserted to avoid ringing and an element inserted to defeat ringing should be able to come into equilibrium around spring curve 35.

FIGS. 4 to 7 illustrate schematically various in-elastic elements, useful for example to suppress generation of ringing or rebound during impulse transfer. In each of the Figures the sketch to the left illustrates the initial state before yield and the right sketch the final state after yield.

In FIG. 4 the element 40 has a front end 41 and a rear end 42 for serial insertion into a force chain. The element comprises a container 43 in the form of a shape permanent cavity containing particles 44, e.g. shots, to a high filling degree. The distance A illustrates the dislocation of particle mass between the initial and final states and can be said to represent roughly the stroke length and possibly also the lost distance since the element may need to be accelerated such a distance to securely locate the particles in the initial state. The distance may also give a corresponding delay at impact, which can be beneficial if adapted to the compression of system elasticities but can be reduced if desired, e.g. by a liquid filling. This element is inherently stiff since the container is solid and accordingly the element can easily transmit even high peak forces.

In FIG. 5 the element 50, with front part 51 and rear part 52, is somewhat similar to that of FIG. 4 although its cavity type container 53 contains only one weight 54, movable over the distance B. In order for the weight not just create impact but continuous counter-force upon retardation the weight is frictionally engaged to the container, here illustrated as a guiding rod 55 along which the weight is frictionally movable. Alternatively the cavity can be liquid filled with clearings 56 between weight and container walls adapted to give suitable damping friction. Certainly the friction can be given an over length variable force profile, e.g. with increasing force. A weak return spring 57 can be present for moving the weight back to the initial state for repeated use, which return spring can be much weaker than the forces involved the injection, e.g. just slightly more than needed to keep weight fixed against gravity in the initial state. Also the container 53 is inherently stiff as in the embodiment of FIG. 4 and the distance B has a similar meaning.

In FIG. 6 the element 60, with front part 61 and rear part 62, is collapsible by comprising a container support 63 containing soft structures 64 that can be crushed or upset to a shorter final state as illustrated to the right where the distance C illustrates the stroke length or lost distance. The container can have a variety of forms from simple supports to full confinement allowing the collapse but is here depicted as two telescoping parts. The structures are naturally stiffening during collapse which replaces the lack of force carrying properties of the container. This element can be designed with low cost materials in the structures, e.g. for diposable purpose, although memory materials can be used for repeated use.

In FIG. 7 the element 70, with front part 71 and rear part 72, is collapsible by being designed as a sand bag although its particulate filling 74 need not be sand but can be other materials as well, e.g. heavy metal shots as in FIG. 4. The container 73 may comprise a resilient membrane or bag 75, possibly pre-stressed, assisting in maintaining a stable form, e.g. rounded, in the initial state but allowing flattening during collapse as illustrated in the right sketch, where the deformation distance D illustrates the stroke length or lost distance. A rigid support 76 can be used to stabilize the element structure and facilitate attachment in the force chain. It is possible to eliminate any physical front part 71 structure and let the bag 75 attack directly on the movable wall, piston rod etc. Also this kind of element is automatically stiffening by increasing contact surface and can be used repeatedly.

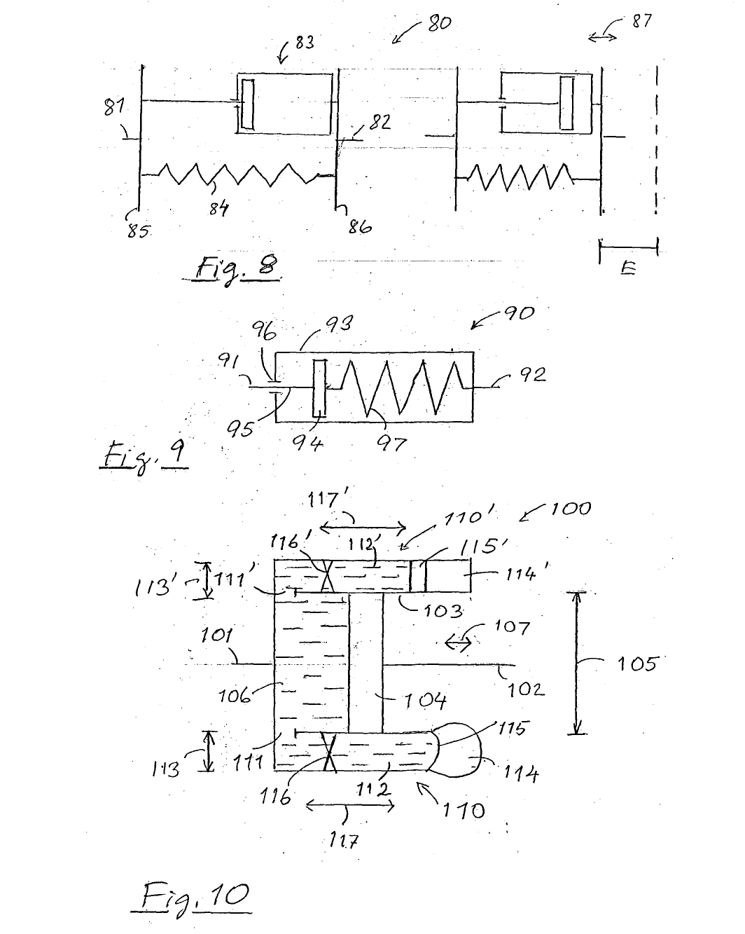

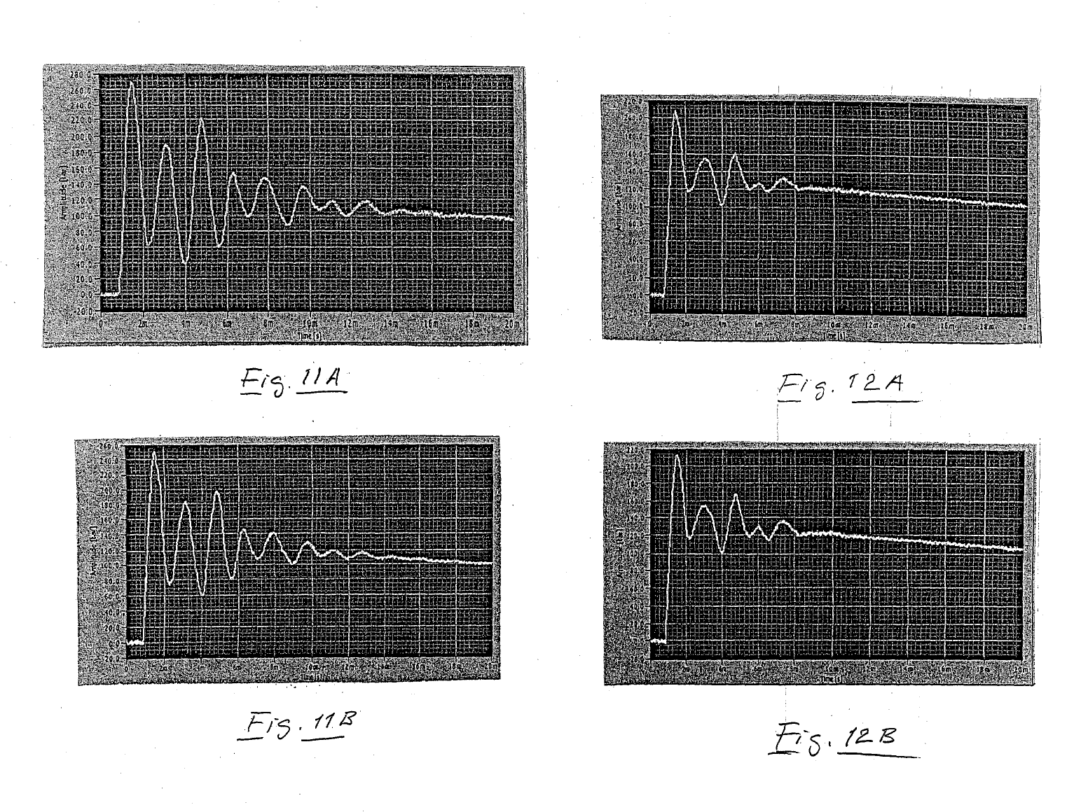

FIGS. 8 to 10 illustrates schematically the principles of a combination element able to come into equilibrium with the applied force in the force chain and useful for example as filter or damper for existing ringing oscillations. Although the dampers are exemplified as viscous cylinder type designs and the springs as coil or gas springs it should be clear that other component types can be used instead. Although, as in the previous Figures, the front parts and rear parts of the elements are schematically shown as lines for connection to a hypothetical force chain it should be understood that any attachment can be used an that the element will operate even if the front and rear parts are reversed.

In FIG. 8 the combination element 80, with front end 81 and rear end 82 as earlier, is shown to have a damper part 83 connecter in parallel with a spring part 84 between common supports 85 and 86 respectively so that the damper and spring will compressed and extended together with similar distances. The strength of spring 84 shall preferably be selected so that it becomes only partially compressed at all force levels present during the injection, which allow the element to come into equilibrium with these forces. The sketch to the left illustrates an initial state under no external compression whereas the sketch to the right illustrates partial compression over the distance E. Assuming that this state represents equilibrium with a force level in the main target profile, the element can still perform oscillations around this force level as indicated by double-arrow 87. The damper 83 acts to defeat or reduce the oscillations, as compared to a non-damped system, by resisting the forces and dissipating the energy therein. The damper can be of any type and any characteristic discussed although here illustrated as a viscous cylinder/piston type assumed to have by-pass arrangements through or around the piston. Similarly the spring can be of any type although here illustrated as a coil spring. The stroke length of the element should be adapted mainly with consideration to the maximum size of the oscillations 87, which in spite of high pressure amplitudes may be quite small when translated into axial distances due to the hard materials involved in system elasticities. If a damper having increasing resistance with displacement speed, like a liquid viscous damper, the element will be quite stiff during the rapid initial pressure rise. It should be noted that the damper 83 and the spring are connected in parallel, in the sense discussed, since the damper is able to retard the action of the spring.

FIG. 9 illustrates schematically a more practical design of a combination element than the principle sketch of FIG. 8. The element 90, with front end 91 and rear end 92, which ends can certainly can be reversed. The damping part of the element is a viscous cylinder 93 piston 94 type wherein the piston is attached to a piston rod 95 passing out of the cylinder and acting as the element front part 91 and being sealed to the cylinder at 96. The cylinder is supposed to be filled with a fluid, preferably a liquid, and to have by-pass arrangements through or around the piston for friction generation. The spring 97 has the same function as spring 84 of FIG. 8 and can be dimensioned according to the same principles. It acts between the cylinder 93 and the piston 94 and in spite its position between these elements it is arranged in parallel, in the present sense, with the damper component as it is able to retard the extension of the spring. This element will act in the same manner as described in connection with FIG. 8.

FIG. 10 illustrates schematically a combination element with arrangements for stroke length amplification with respect to lost distance. The element 100, with front part 101 and rear part 102, comprises a main cylinder 103 with a main piston 104, acting as rear part 102 of the element and sealingly engaged to the walls of the main cylinder 103, acting as front part 101 of the element. The main piston 104 has a large cross-section area, illustrated with the long arrow 105. The interior of main cylinder is filled with a liquid 106. The interior of the main cylinder is connected to a combination element, here illustrated with two alternatives 110, and 110′ respectively intended to be used as alternatives. Both combination elements have a liquid connection 111 or 111′ to the liquid 106 in the main cylinder, a channel 112 or 112′, the cross-section area of which, illustrated with arrows 113 or 113′, is smaller than the cross-section area of the main cylinder, as illustrated with arrow 105. Both combination elements further have a spring element in the form of a gas accumulator 114 or 114′, separated from the liquid with a resilient membrane 115 or a movable piston 115′ respectively, and a fixed constriction 116 or 116′ for friction generation against liquid passing the constriction. Movement of main piston 104 in the main cylinder 103 will displace liquid 106 back or forth through connections 111 or 111′ and into channels 112 or 112′ under volume change of gas accumulators 114 or 114′, movement of membrane 115 or piston 115′, and under dissipation of energy in constrictions 116 or 116′. The relationship between main piston 104 cross-section 105 and channels 112 or 112′ cross-sections 113 or 113′ will transform a small main piston oscillation movement, as illustrated with arrow 107, into a larger liquid movement, as illustrated with arrows 117 or 117′, in the channels 112 or 112′ of the combination elements. This amplifies dampening and makes it more reliable, which is of particular value in the present context as the hard elastic components in the system makes even large pressure variations correspond to fairly small displacement variations. Except for the stroke length transformation this combination element will behave as those of FIGS. 8 and 9, e.g. the gas springs will enable the element to come into equilibrium with a target pressure profile force and perform damped oscillations- around- such an equilibrium value.

FIGS. 11A and 11B show two pressure profile samples obtained with a prior art jet-injector pressure chamber, substantially as described in U.S. Pat. No. 5,704,911, incorporated by reference herein. The pressure chamber was a plastic cylinder with an inserted plastic rod having a front piston. The chamber was filled with 0.3 ml water, representing about full nominal filling. The rod was attacked over a gap by a plunger driven by a coil spring. A sensor inserted laterally through the chamber wall measured the pressure in the chamber. The recordings show the pressure in bar units against time in seconds. For resolution reasons the time scale shows only the first 20 milliseconds of the injection. It is clear that a significant ringing is superimposed on the injection part of the curve, which ringing seems to even out after about 14 milliseconds.

FIGS. 12A and 12B show two pressure profile samples of the jet-injector of the same jet-injector as used in FIG. 11, although the plunger of the mechanism was modified with a collapsible element of the type earlier described, having a shape permanent cavity filled to about 90% bulk volume with metal shot particles. The recordings were made as described in connection with FIGS. 11A and 11B. Although the modified plunger was not optimized for its purpose, it is clear that the ringing is less pronounced and evens out earlier at about 8 milliseconds.

1. A jet-injector device comprising a) a housing, b) a pressure chamber for a liquid to be ejected attached to or enclosed in the housing, the pressure chamber having at least one opening and at least one movable or collapsible wall or wall segment and c) a pressurizing mechanism attached to or enclosed in the housing operable to apply, directly or indirectly, force in a force chain between the housing and the wall to pressurize the pressure chamber content for ejection of a liquid jet through said opening, the mechanism comprising at least a force generator and optionally a transmission between the force generator and the wall, characterized in the improvement comprising, an in-elastic element serially arranged between the force generator and the wall.

2. The device of claim 1, wherein the element is designed to remove work energy forms, at least partially irreversibly, from force applied to at least two parts of the element displaceable, externally or internally, with respect to each other.

3. The device of claim 2, wherein the element is designed to remove work with a friction mechanism.

4. The device of claim 1, wherein the element comprises a viscous damper component.

5. The device of claim 1, wherein the element comprises a mechanical friction component.

6. The device of claim 5 wherein the element comprises a deformable inelastic component.

7. The device of claim 6, wherein the element comprises a collapsible container part.

8. The device of claim 6, wherein the element comprises a shape permanent container.

9. The device of claim 8, wherein the container comprises at least one movable mass.

10. The device of claim 9, wherein the container comprises particles to a filling degree of at least 50% bulk volume.

11. The device of claim 1, wherein the element has a minimum stroke length, when measured at the wall, of at least 1 mm, preferably at least 2 mm and most preferably at least 3 mm.

12. The device of claim 1, wherein the element comprises a stroke length limiter.

13. The device of claim 12, wherein the limiter provides an over length increasing counterforce.

14. The device of claim 13, wherein the limiter comprises an elastic component.

15. The device of claim 14, wherein the limiter is arranged to allow the element to come into equilibrium with different forces transmitted during operation of the device.

16. The device of claim 1, wherein the element has an internal ratio between external and internal stroke lengths.

17. The device of claim 16, wherein the ratio is arranged-to amplify internal stroke length.

18. The device of claim 1, wherein the element has a force ratio, as defined, of less than 100%, preferably below 90%, below 75%, below 50% or below 25%.

19. The device of claim 1, wherein the element is arranged in the interface between hard and soft force chain parts, as defined.

20. The device of claim 1, wherein the element is positioned is in the front-most part of the force chain at or close to the pressure chamber movable wall, or a piston rod for the wall.

21. The device of claim 1, wherein the element is arranged for the purpose of damping out existing ringing.

22. The device of claim 21, wherein the element is arranged to allow repeated strokes.

23. The device of claim 22, wherein the element is a combination element of at least one in-elastic component and at least one elastic component.

24. The device of claim 1, wherein the element is arranged for the purpose of avoiding rebound effects at impact.

25. The device of claim 24, wherein the impact results from a gap in the force chain.

26. The device of claim 25, wherein the element is a combination element of at least one in-elastic component and at least one elastic component.

27. The device of claim 26, wherein the element is a deformable element.

28. The device of claim 1, wherein the mechanism is arranged to give an initial penetrating peak force followed by a lower injection force.

29. The device of claim 28, wherein the elements is arranged to provide an initial counter-force.

30. The device of claim 29, the element is arranged not to yield substantially below forces corresponding to at least 10% of the maximum force value in the initial peak, preferably not below 20% and most preferably not below 30% of this force.

31. The device of claim 29, wherein the element is arranged to yield below the maximum peak value, preferably below 90% and most preferably below 80% of this value.

32. The device of claim 31, wherein the element is arranged to begin yielding roughly at a force value where a force line for the main spring intersects with the initial peak.

33. The device of claim 32, wherein the element is arranged to increase its counterforce on displacement of its parts.

34. The device of claim 32, wherein the element is arranged to allow increased counter-forces at least to forces corresponding to the maximum force value in the peak

35. The device of claim 28, wherein the counterforce comprises a resistance force of the element in-elastic component, disregarding any elastic element component, the resistance force being above 10%, preferably above 20% and most preferably above 30% of the maximum peak force and is below 90%, preferably below 80% and most preferably below 60% of the maximum peak force.

36. The device of claim 1, wherein the element is arranged to provide a resistance force of the element in-elastic component, disregarding any elastic element component, the resistance force being above 10%, preferably above 20% and most preferably above 30% and is below 90%, preferably below 80% and most preferably below 60% of the momentary force transmitted in the force chain.

37. Use of the device according to any of claims 1 to 36 to prevent aspiration pressures in the pressure chamber.

38. A method for generation of a high speed liquid jet, the method comprising the step of pressurizing the liquid when in a pressure chamber, having at least one opening for the liquid jet and having at least one movable or collapsible wall or wall segment, by applying a pressurizing force on the wall, characterized in the improvement comprising the steps of i) applying a primary force, directly or indirectly, on one part of an in-elastic element, ii) applying the pressurizing force by another part of the element, to thereby press the element between the primary force and the pressurizing force and iii) dissipating energy in the element.

39. The method of claim 38, including the step of accelerating a mass with the primary force.

40. The method of claim 39, wherein the accelerating step takes place before applying the pressurizing force.

41. The method of claim 38, including the step of squeezing the element between the primary force and a support prior to applying the pressurizing force.

42. The method of claim 41, wherein the squeezing step comprises the step of allowing the element to respond with a counterforce substantially in equilibrium with the primary force.

43. The method of claim 38, wherein the dissipating step comprises the step of allowing the element to oscillate.

44. The method of claim 38, wherein the dissipating step comprises the step of allowing the element to collapse.

45. The method of claim 38, wherein the dissipating step comprises the step of allowing the element to change mass center.

46. The method of claim 38, wherein the element is designed according to any characteristic of claims 1 to 37.

47. Use of the method according to any of claims 38 to 46 to prevent aspiration pressures in the pressure chamber.

48. A jet-injector having a pressure chamber for a liquid and a mechanism for pressurizing the liquid in the pressure chamber, the jet-injector being operable to perform injections without aspiration pressures in its pressure chamber.
